(19) United States
(12) Patent Application Publication
(10) Pub. No.: US 2010/0280313 A1
(43) Pub. Date: Nov. 4, 2010

(54) SYSTEMS AND METHODS FOR ENDOSCOPIC INVERSION AND REMOVAL OF DIVERTICULA

(75) Inventors: Christoph Gasche, Klosterneuburg (AT); Stephen Graham Bell, Roma (IT); Wayne A. Noda, Mission Viejo, CA (US); Bradley J. Sharp, Irvine, CA (US)

Correspondence Address: ROGITZ & ASSOCIATES, 750 B STREET, SUITE 3120, SAN DIEGO, CA, 92101, US

(73) Assignee: Minos Medical

(21) Appl. No.: 12/831,620

(22) Filed: Jul. 7, 2010

Related U.S. Application Data
(62) Division of application No. 11/788,597, filed on Apr. 20, 2007.

(51) Int. Cl. A61B 17/12; A61B 1/00

(52) U.S. Cl. 600/104; 606/153; 606/32; 606/219; 606/213

(57) ABSTRACT

Systems and methods are disclosed for the inversion of gastro intestinal diverticula and repair of associated intestinal wall tissue by means of endoscopy through a natural orifice such as the mouth or anus without making incisions in the abdominal wall or opening the peritoneal cavity.

### I. FIELD OF THE INVENTION

The present invention relates generally to systems and methods for transanal and transoral endoscopic inversion of diverticula and repair of intestinal wall defects that cause the diverticula.

### II. BACKGROUND OF THE INVENTION

Diverticulosis is an unfortunately common condition in which an area of the intestine bulges out into the peritoneal cavity to form a sac referred to as a “diverticulum”. Diverticula are also formed when a point of weakness in the intestinal wall, e.g., where blood vessels take their entry, causes the muscular structure to divide and allows part of the inner mucosal membrane to be pushed out of the hole into the peritoneal cavity and become filled with fecal matter. Diverticula may also be present in other parts of the gastrointestinal tract such as the esophagus or the duodenum.

Symptoms may be mild and intermittent or acute and severe. In the latter case, the disease is usually caused by inflammation of the colonic wall and/or the pericolic fat. In this sense, diverticulitis requires both the presence of diverticula and inflammation by definition. Inflammation is often caused by impacted fecal matter or faecoliths. In other words, diverticular disease frequently arises due to the intestinal outpouchings (the diverticula) becoming blocked with feces, allowing bacteria to build up, causing infection.

On a first admission with acute diverticulitis, the majority of patients respond to medical treatment (i.e., antibiotics), with the remainder requiring surgery. Those requiring surgery have a non-inconsequential mortality rate exceeding ten percent, and in the event that a diverticulum is itself perforated the mortality rate rises. Of the patients who respond to antibiotics, many experience at least one recurrence of diverticulitis with decreasing response to medical therapy and increasing requirement for surgical resolution.

Accordingly, regardless of its cause, it happens that many patients suffering from diverticulitis require surgical intervention with its attendant risks. To this end, open or laparoscopic colonic segmental resection is the current method of choice, not an easy chore not least because it can be difficult to locate diverticulanested in the pericolic fat.

Thus, since locating all of the diverticula in the pericolic fat is problematic and because much of the disease typically is associated with the sigmoid colon, the surgical strategy especially when preventing recurrent disease is to remove the diseased colonic segment. As understood herein, however, the colorectal junction area is sometimes alluded to as a high pressure zone, meaning that this area should be resected as well because when it is left behind recurrence is increased by many times. Thus, the current general perception is that the anastomosis should always be to the rectum below the peritoneal fold.

As understood herein, removing so much of a patient's intestine is less than optimal, since the intestinal length is designed by nature to be as long as it is. Moreover, the 30-day complication rate from the above-described surgery may exceed twenty percent, with a measurable 30-day mortality rate and reoperation rate.

U.S. Pat. No. 5,100,419, incorporated herein by reference, discloses a method for advancing an endoscope into the colon and resolving diverticula from the inside by using vacuum to invert them back into the scope apparatus, where they are closed using rubber bands. As understood by the present invention, while the '419 patent offers an alternative method for treating diverticulitis that does not require anastomosis, it leaves the banded-off diverticula in the colon, depending on the diverticula to eventually slough off and pass through the colon. The present invention understands that it is desirable to maintain more positive control over both the removal of diverticula from the body, and ensuring that the colonic wall remains structurally sound, to prevent recurrences. The invention is also directed to addressing diverticula in other parts of the gastrointestinal tract that can be accessed through natural orifices.

### SUMMARY OF THE INVENTION

Using an endoscope referred to as a colonoscope that is advanced through a natural orifice such as the anus or esophagus, diverticulitis is treated by first inverting the diverticular tissue (called a “diverticulum”) into the intestinal passage, repairing the inverted tissue with trans-muscular sutures, then in some embodiments either cutting it off with a cautery wire or other cutting element such as a blade and retracting it through the instrument. Alternatively, the diverticulum can be left in place to slough off after wall repair. In any case, a suture can be placed through the muscular layer (to attach serosa to serosa on the outside) to thereby close the muscular leak that caused the defect in the first place. This suture is also placed using the instrument. Other means for resolving the deep tissue defect are also disclosed.

Accordingly, a method is disclosed for treating a diverticulum formed in an intestinal wall of a patient. The method includes advancing a tubular assembly through a natural orifice into the intestine to the site of the diverticulum, and drawing the diverticulum into the assembly. The method also includes resolving a defect area associated with the diverticulum at least in part using the assembly by attaching one portion of the entire muscular wall of the intestine to another portion of the entire muscular wall. An “entire wall” is composed of a mucosal-submucosal-muscular-serosal layer, from inside to out.

In some implementations the defect area is resolved by drawing the diverticulum into the assembly, removing the diverticulum, and holding together intestinal wall tissue serosa to serosa, it being understood that holding tissue serosa to serosa optimized healing. For example, the intestinal wall tissue can be sutured or stapled to hold it together serosa to serosa. Or, the intestinal wall tissue can be held together using a barbed anchoring device. Yet again, the intestinal wall tissue may be held together serosa to serosa by clamping the wall between opposed arms of a tissue heating device and heating the arms to fuse clamped wall portions together. In other aspects, the defect area can be resolved by disposing a cover over the defect area and adhering the cover to the intestinal wall. In still other aspects, a plug is used to plug the defect area.

In another aspect, an overtube assembly includes an overtube configured for advancement through a natural orifice such as the anus into the colon of a patient. Means are engageable with the overtube for inverting a diverticulum into the overtube. Also, means are engageable with the overtube for resolving a defect area associated with the diverticulum by closing a defect in a muscular wall of the colon.

In yet another aspect, an assembly for removing diverticula and resolving associated defect areas includes an overtube advanceable into the colon of a patient, and an inversion component engageable with the overtube and juxtaposable with a diverticulum to invert the diverticulum into the colon. A ligator is engageable with the overtube and is operable to ligate the diverticulum. A defect'resolution mechanism engages the overtube for resolving an intestinal wall defect associated with the diverticulum.

The details of the present invention, both as to its structure and operation, can best be understood in reference to the accompanying drawings, in which like reference numerals refer to like, parts, and in which:

### BRIEF DESCRIPTION OF THE DRAWINGS

FIG. 1 is a schematic view showing the tube assembly including overtube, colonoscope, and optional steering catheter being advanced through the anus into the colon;

FIG. 2 is a schematic view showing the tube assembly advanced to a diverticulum, with the open end of the overtube positioned around the diverticulum;

FIG. 3 is a schematic view showing an evacuation catheter advanced into the diverticulum, and also showing the proximal and distal vacuum seals, the colonoscope omitted for clarity;

FIG. 4 is a schematic view showing the diverticulum inverted into the overtube and a prettied ligature loop advanced around the diverticulum, prior to tightening the ligating device;

FIG. 5 is a schematic view showing the diverticulum inverted into the overtube and showing an alternate embodiment in which a suture needle is shown poised to be passed through the tissue;

FIG. 6 is a schematic view showing the diverticulum inverted into the overtube and the needle passed through the tissue and impacted into the receiving plug;

FIG. 7 is a schematic view showing the diverticulum inverted into the overtube and the plug with needle being retrieved to pass a suture through the diverticulum;

FIG. 8 is a schematic view showing the diverticulum inverted into the overtube and tied shut using a knot pushing device to advance half hitch knots to the suture site;

FIG. 9 is a schematic view showing the inverted and ligated diverticulum with an electrocautery polypectomy snare advanced around the suture site to transect the diverticulum;

FIG. 10 is a schematic view showing a mechanical grasper for inverting a diverticulum;

FIGS. 10A and 10B are schematic views showing a bristle brush for inverting a diverticulum;

FIGS. 11-15 are schematic side views showing a first embodiment of resolving the intestinal wall defect after the diverticulum has been transected, using a cover patch;

FIGS. 16-21 are schematic side views showing a second embodiment of resolving the intestinal wall defect after the diverticulum has been transected, using staples;

FIGS. 22-27 are schematic side views showing a third embodiment of resolving the intestinal wall defect after the diverticulum has been transected, using a barbed T-shaped anchor;

FIGS. 28-31 are schematic side views showing a fourth embodiment of resolving the intestinal wall defect after the diverticulum has been transected, using heat sealing jaws;

FIGS. 32-36 are schematic side views showing a fifth embodiment of resolving the intestinal wall defect after the diverticulum has been transected by pulling one part of the wall over another and fastening the two parts together using a T-shaped anchor;

FIGS. 37-42 are schematic side views showing yet another alternate embodiment in which a hollow plug is first advanced into the diverticulum, with the diverticulum being inverted through the plug, removed, and the plug remaining in place to resolve the colonic wall defect;

FIGS. 43-48 are side views of an alternate plug; and

FIGS. 49 and 50 are side views of still another alternate plug.

### DETAILED DESCRIPTION OF THE PREFERRED EMBODIMENT

Referring initially to FIG. 1, a catheter assembly is shown, generally designated 10, that includes a flexible hollow overtube 12 fixedly or slidably holding one or more components such as but not limited to a colonoscope 14 and a steering catheter 16. The colonoscope 14 may extend from the open distal end 18 of the overtube 12 as shown to a colonoscope control hub 20 that is external to the patient. In this way, for example, images of the colon 22 from the colonoscope 14 can be presented on a monitor to a surgeon. Other components in the overtube 12 may extend to other hubs, e.g., the steering catheter 16 may extend to manipulable control hub 24 for turning and directing the steering catheter 16 (and, hence, the overtube 12) under visualization provided by the colonoscope 14. The overtube 12 itself may have steering capability my means of, e.g., anchoring a steering wire in the overtube 12 near the distal end 18, with the steering wire being manipulable to bend the overtube 12.

The components 14, 16 may extend through respective working lumens of the overtube 12. Additional components may extend through additional working lumens and/or may be swapped according to the stage of the procedure for the components 14, 16. As described further below, the additional components may include catheters for inverting diverticula into the intestinal lumen, transmural suturing/clipping devices, detachable ligating devices, and polypectomy snares. Thus, in non-limiting implementations the overtube 12 may include additional lumens with respective proximal and distal openings.

As intended herein, the overtube 12 can be flexible and can be sized as appropriate for the patient, e.g., shorter overtubes can be provided for pediatric patients. Although not shown in FIG. 1, the open distal end 18 of the overtube 12 may be tapered into a frusto-conical shape to reduce the risk that parts of the colon wall may become trapped between the overtube 12 and the colonoscope 14 during advancement of the overtube 12. Also, the inner surface of the overtube 12 can be coated with a lubricious material to ensure smooth advancement of the overtube 12 over the colonoscope 14.

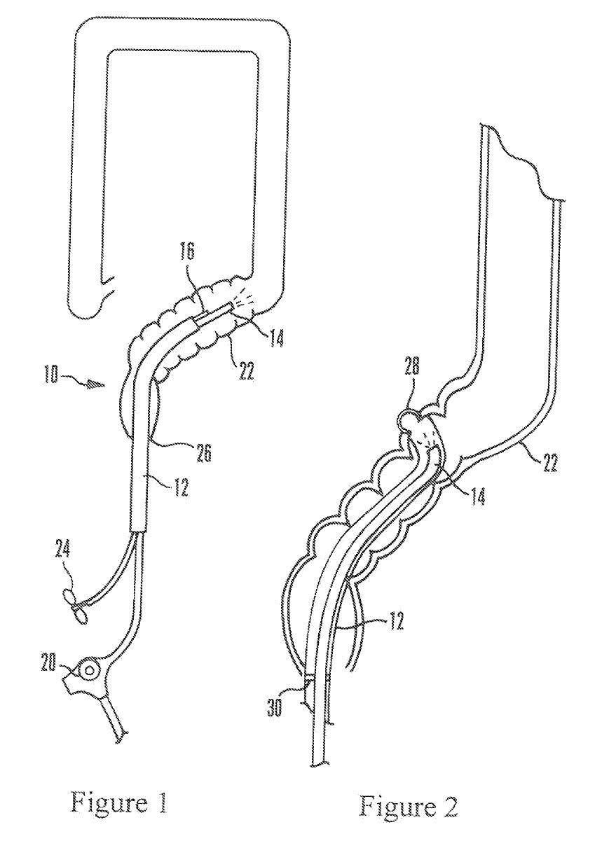

Prior to discussing details of various structures for resolving defect areas associated with diverticula, FIGS. 2-9 first will be described to give both an overview of one example procedure in accordance with present principles, as well as non-limiting structure that can be used to undertake the procedure. As shown in FIG. 2, the catheter assembly 10 is advanced through the anus 26 into the colon 22 to an area of diverticular disease, i.e., to a diverticulum 28. Due to the nature and location of the diverticulum 28, the assembly 10 may be guided by colonoscopy. Alternatively, the catheter can be advanced into the small intestine through the esophagus, in which case upper GI imaging and enteroscopy may be used to guide the catheter.

The overtube 12 is typically advanced together with the colonoscope 14. When the distal end 18 of the overtube 12 is kept behind the tip of the colonoscope 14, the colonoscope 14 has full maneuverability. During colonoscopy, air or some other gas may be used if desired to insufflate the otherwise collapsed colon. A flexible seal 30 can be provided in the proximal part of the overtube 12 to prevent insufflation air from leaking between the colonoscope 14 and overtube 12. When the air or the gas is removed by suction through a channel such as an instrument channel of the colonoscope 14, this seal 30 also ensures the maintenance of vacuum in the overtube 12 for inversion of the diverticulum 28 as will be shortly discussed.

To enhance locating diverticula, a barium study can first be employed to ascertain the number and location of diverticula prior to diverticuloectomy. Alternatively, contrast media can be flushed into the colonic lumen during colonoscopy.

Turning to FIG. 3, once the overtube 12 has been positioned adjacent to a diverticulum 28, the steering catheter 16 if not already positioned may be advanced through a working channel of the overtube 12 to the diverticulum 28 as shown. The catheter 16 may include its own working lumens such that saline can be infused into the diverticulum 28 to flush out fecal matter for later removal through, e.g., the instrument channel of the colonoscope 14. At this point of the procedure, acute diverticulitis can be diagnosed by the appearance of purulent material at the diverticular orifice.

If desired, contrast media can be infused through the catheter 16 to facilitate obtaining radiographs from the diverticulum 28. In this way, the size and location of the diverticulum 28 can better be seen using fluoroscopy, to check the diverticulum 28 for signs of fistulization or perforation. If the diverticular orifice shows a stricture or does not have an adequate diameter for inversion, it may be dilated using an inflatable balloon or similar structure that can be engaged with the catheter 16.

Next and cross-referencing FIGS. 3 and 4, the open distal end 18 of the overtube 12 preferably is provided with a distal seal 32 for sealing against the intestinal wall (FIG. 3), and then a vacuum is drawn through the overtube 12 to invert the diverticulum 28 (FIG. 4). In lieu of using the entire overtube 12 the catheter 16 or another catheter such as the colonoscope 14 may be used for evacuation purposes. Regardless of which catheter is used, the diverticulum 28 is pulled into the overtube 12 in the intestinal lumen.

The distal seal 32 of the overtube 12 may be established by spikes, rings, wires, balloons, forceps, flaps, high friction surfaces, glue, or any combination thereof. In non-limiting implementations the distal seal 32 can be moved into position after the overtube 12 has been navigated into position with the colonoscope 14. For example, a tether can be pulled to draw the seal 32 into place to provide a vacuum seal.

It may now be appreciated that the overtube 12 produces counterforce around the orifice of the diverticulum 28 as the diverticulum is inverted. This causes the diverticulum 28 to partially or completely invert into the intestinal lumen. Vacuum is built up in the space of inversion and is sealed from leaking to the intestinal lumen by the distal seal 32 of the overtube 12. As stated above, not only may the vacuum be established and/or maintained by applying suction through the catheter 16, it may also be established/maintained by applying suction through the colonoscope 14 or through a separate suction lumen of the overtube 12.

Once the diverticulum 28 is inverted, FIG. 4 illustrates that it may be ligated. In one non-limiting implementation, a preloaded string or detachable loop 34 such as a prettied ligature loop is disposed on the overtube 12 at the distal end 18 thereof such that the diverticulum 28 is inverted through the loop. The string 34 extends through the overtube 12 as shown so that it may cinched tight around the diverticulum 28.

Alternatively, a transmural suture or clipping device can be placed at the distal end 18 of the overtube 12. The transmural device has the ability to close the opening in the muscular layer of the bowel wall and to prevent recurrence of a diverticular pouch.

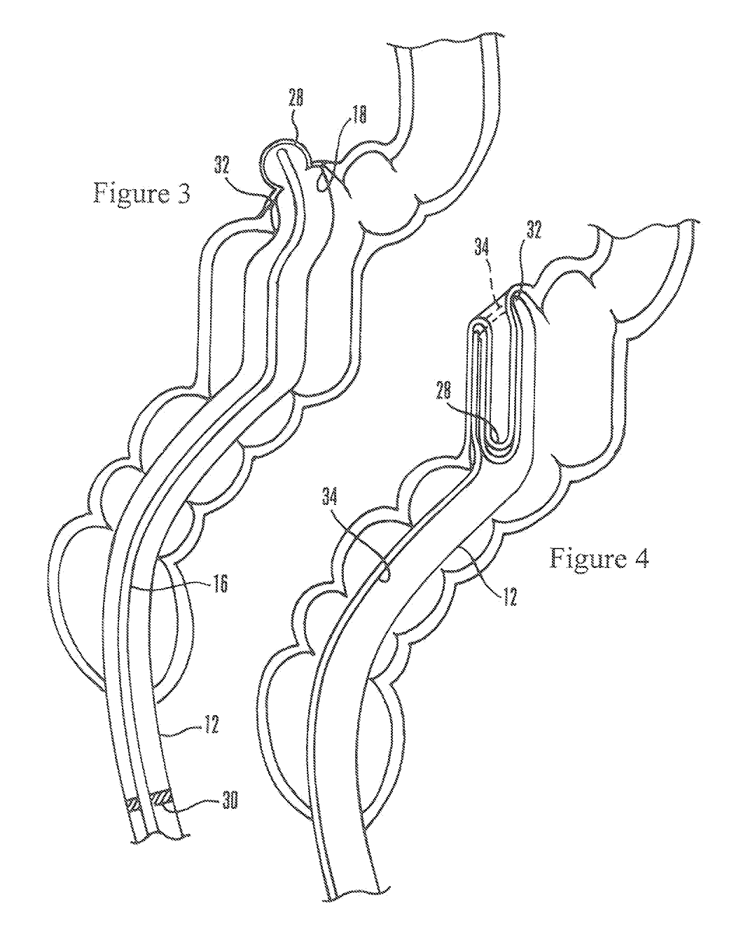

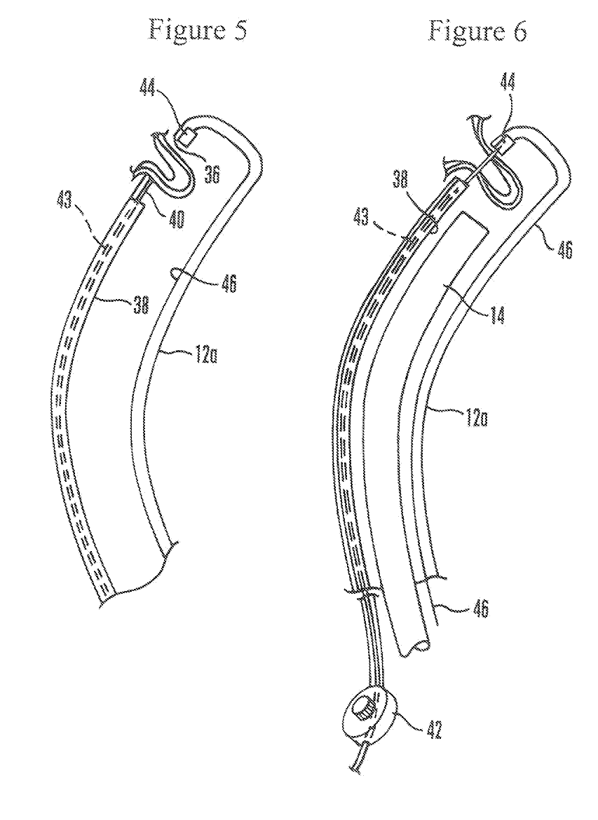

FIGS. 5-9 show remaining steps of the procedure with alternate ligating structure. For completeness of disclosure FIGS. 5-9 show an alternate overtube 12a that has a side opening 36 in lieu of or in addition to an open distal end (which otherwise can be sealed off if desired with, e.g., a balloon), it being understood that the overtube 12 shown in FIGS. 1-4 equally may employ the following principles.

As shown best in FIGS. 5 and 6, a needle catheter 38 holding one or more preloaded suture needles 40 may be disposed in the overtube 12a as shown. Equivalently, a lumen of the overtube 12 can be used to deliver the needles 40. The needles 40 can be pushed distally using an external pusher handle 42 (FIG. 6) or other structure. For example, the needles 40 can simply be nested into the end of a pushrod since all the required pushing force is in one direction. Each needle 40 is attached to opposite ends of a single length of suture string 43.

A plug 44 of material such as urethane foam, rubber, etc. is disposed on the opposite side of the side opening 36 from the needles 40 as shown. With this structure, the needles 40 can be pushed through the diverticulum 28 into the plug 44, which captures the needles 40 after penetrating the tissue, as shown in FIG. 6. If desired, the needles 40 may be barbed to enhance their pullout strength.

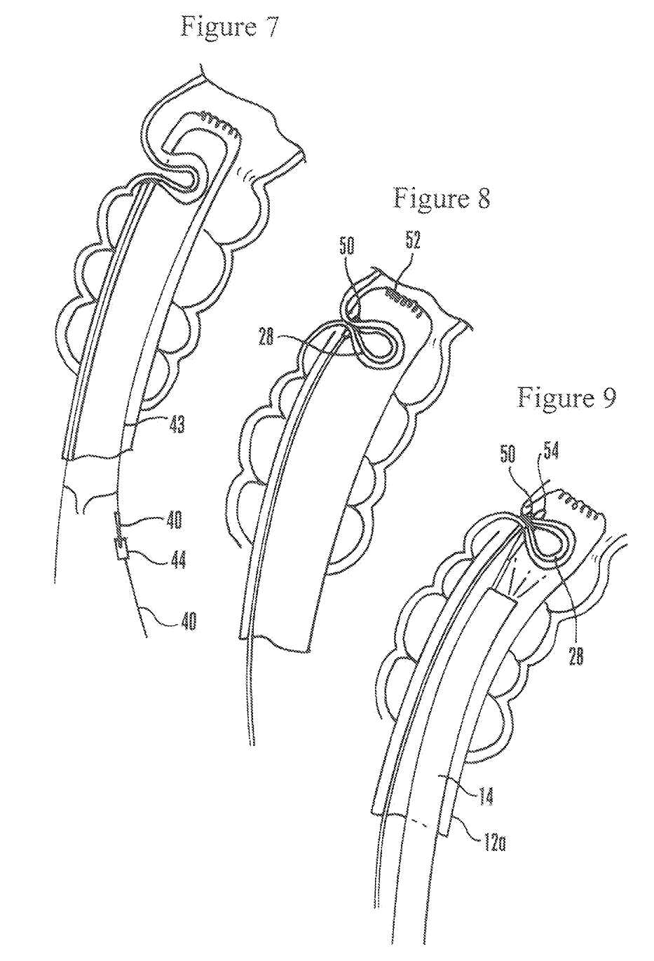

The plug 44 is attached to a retrieval string 46 that extends through a lumen of the overtube 12 as shown in FIG. 6. The plug 44 with embedded needles 40 is then retrieved by tensioning the retrieval string 46 (FIG. 7) to thereby pull the suture string 43 through the diverticulum 28 and back out to the operator. The operator can then use a knot pushing device to advance half hitch knots 50 to the suture site in accordance with suture principles known in the art (FIG. 8). Alternatively, a crimpable sleeve or compression sleeve can be advanced over the suture tails to secure the closure. If desired, in addition to the distal seal 32, when the overtube 12a has an open distal end in addition to the side opening 36, a closure or seal 52 can be provided on the open distal end as shown. If desired, the diverticulum 28 with associated bowel wall can be twisted prior to suturing to improve closure. In this embodiment the intestinal wall is resolved by a serosa to serosa closure.

Next, as shown in FIG. 9 an electrocautery polypectomy snare 54 can be advanced under visualization provided by the colonoscope 14 through a working channel of the colonoscope. The snare 54 is positioned around the ligated diverticulum 28 as shown and actuated to transect the diverticulum 28 using electrocut and/or electrocoagulation. The colonoscope 14 may then be used to retrieve the diverticulum 28 by pulling it through the overtube 12a using, e.g., forceps that are advanced through the colonoscope 14 or overtube 12a. The overtube can be kept in place and used for fast reinsertion of the colonoscope 14 for the purpose of a second look at the site of colonoscopic diverticuloectomy. Several diverticula can be removed within a single endoscopic procedure. In some embodiments, the diverticula may be simply ligated and left in place to slough off later through the colon.

As shown in the above figures and as discussed more fully below, serosa to serosa closure preferably is effected, in which the entire muscular wall of the colon is inverted and sutured. This closes the muscular leak that caused the primary defect for the diverticulum 28 to be formed in the first place, minimizing recurrences of diverticular disease at the same site.

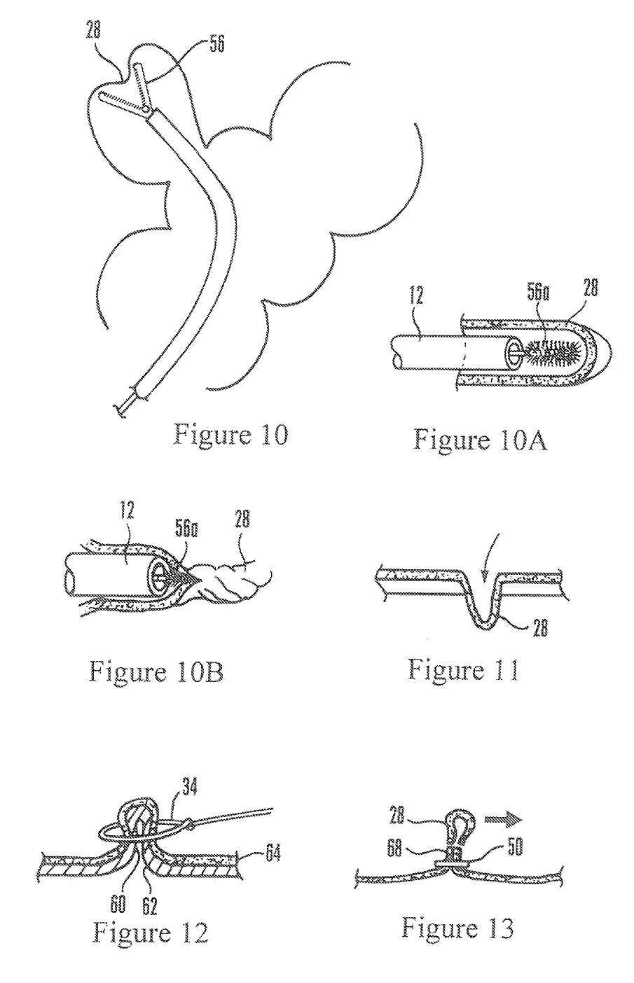

FIG. 10 shows that in lieu of inversion using vacuum, a mechanical grasper or hooking device 56 can be advanced through the overtube 12 or 12a and into the diverticulum 28 to grasp the tissue of the diverticulum 28. The grasper 56 is then retracted into the overtube to invert the diverticulum 28, which can then be ligated and transected in accordance with above principles. Alternatively, as shown in FIG. 10A a bristle brush 56a may be advanced through the overtube 12 or 12a and into the diverticulum 28. A vacuum may be applied through the overtube to suck the tissue against the bristles of the brush, which then is rotated as shown in FIG. 10B to further grasp the tissue of the diverticulum 28, after which the brush 56a may be retracted into the overtube to invert the diverticulum.

The above approach of evacuating the entire overtube to effect inversion may also be used in natural orifice appendectomy procedures or other natural orifice procedures in the gastrointestinal tract.

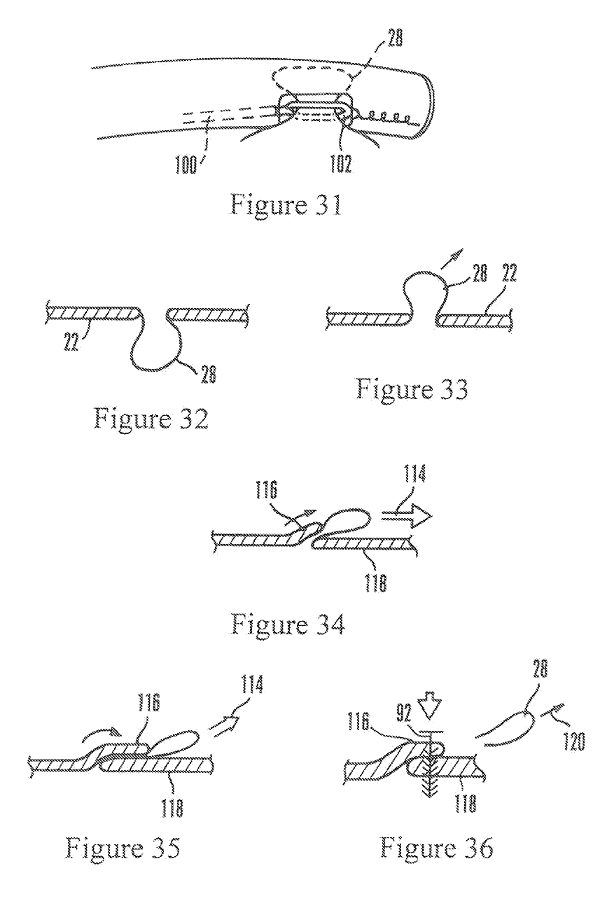

FIGS. 11-50 show the details of various mechanisms for effecting deep tissue closure of the diverticular area after removal of the diverticulum, it being understood that the various mechanisms shown and discussed below can be advanced to the affected site using the catheter assembly 10.

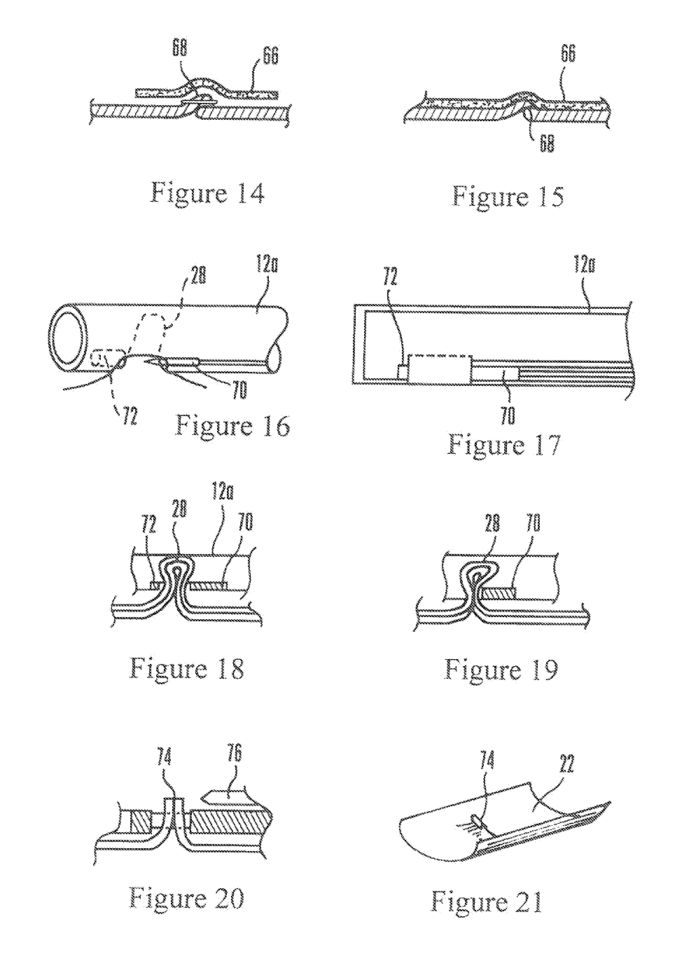

FIGS. 11-15 show that the diverticulum 28 (FIG. 11) can be inverted, ligated with the loop 34 (FIG. 12), tied off using the knots 50 and removed (FIG. 13), with the remaining muscular tissue area of the colon being tied serosa to serosa. In other words, as best shown in FIG. 12 the colonic wall is pursed serosa to serosa, with what had been the outer surface portion 60 of the wall on one side of the diverticulum 28 being flush against the outer surface portion 62 of the wall on the opposing side of the diverticulum 28 to form the pursed serosa-to-serosa structure shown in FIG. 13. Element 64 in FIG. 12 is the inner mucosal membrane or lining of the colon. Once the diverticulum 28 has been removed, a patch 66 is positioned over the stump 68 of the colonic wall and if desired adhered to the colonic wall using a cyanoacrylate as shown in FIGS. 14 and 15.

FIGS. 16-18 show that the diverticulum 28 can be inverted into the overtube 12a between a staple cartridge 70 and an opposed staple anvil 72. The staples can be ejected from the cartridge 70 in accordance with staple ejection principles known in the art to close off the diverticulum 28 as showman FIG. 19, preferably with the muscular wall of the colon pursed as shown in FIG. 20 to form a stump 74. A cutter 76 optionally may be used to cut off the diverticulum, which is removed as described previously, to leave the pursed stump 74 shown in FIG. 21.

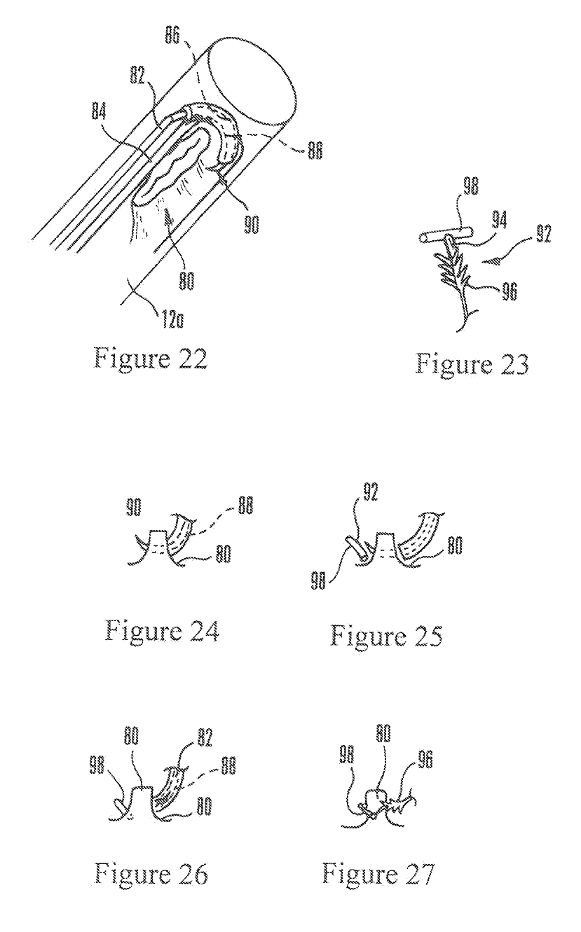

FIGS. 22-27 show another structure for resolving the deep muscle defect associated with the diverticulum 28 after optional removal of the diverticulum leaves a pursed stump 80. A hollow elongated needle tube 82 extends through the overtube 12a and may be supported by an elongated support rod 84 near its distal end as shown. The distal end 86 of the needle tube 82 is curved as shown, and a curved cinch tube 88 slidably surrounds the distal end 86 and is movable my means of reciprocating the support rod 84. A hollow curved needle 90 protrudes out of the distal end 86 of the needle tube 82.

The needle 90 contains one or more barbed “T”-shaped anchors 92 (FIG. 23). As set forth further below, the anchors 92 are delivered through the needle 90 into the stump 80. As shown in FIG. 23; each anchor 92 may include a shank 94 having barbs 96 formed thereon. A cross-bar 98 is formed on one end of the shank 94, and the anchor 92 is made of material that is sufficiently resilient that the bar 98 can be bent parallel to the shank 94 when housed in the needle 90, owing to material bias assuming the shape shown in FIG. 23 once released from the needle 90.

With the above description in mind, as shown in FIG. 24 the needle 90 is passed through the stump 80. FIG. 25 shows that an anchor 92 is ejected through the needle 90 cross-bar 98 first. Then, as shown in FIG. 26 the cinch tube 88 is slid against the stump 80 to capture the stump 80 between the cross-bar of the anchor 92 and the cinch tube 88 while the needle tube 82 is retracted. The barbs 96 of the anchor 92 engage the stump 80 once the cinch tube 88 is retracted as shown in FIG. 27 to trap the stump 80 between the cross-bar 98 and the barbs 96. Proximal barbs 96 may be snipped off or otherwise trimmed away once the anchor 92 is in place. As stated above, the serosa-to-serosa closure may be effected after removing the diverticulum or without removing the diverticulum.

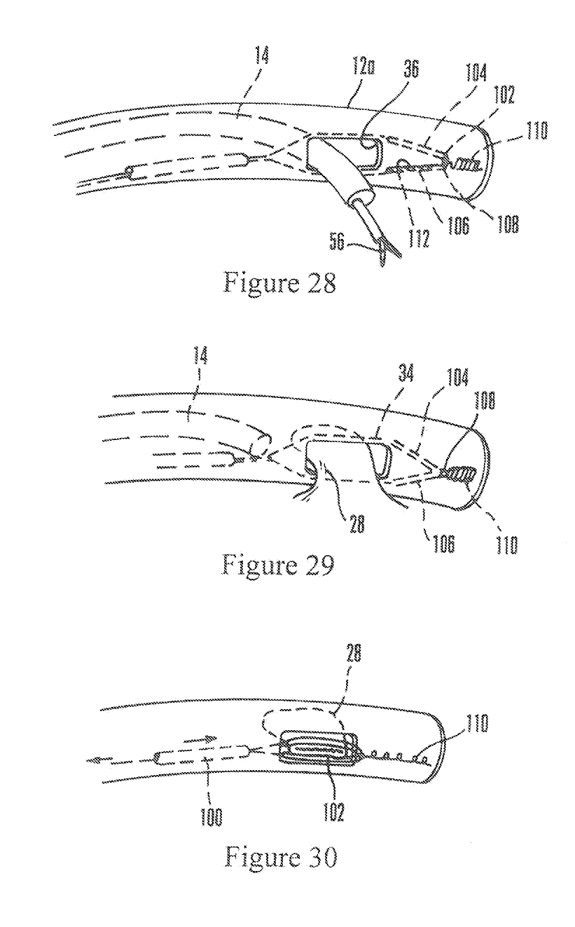

FIGS. 28-31 show that the mechanical grasper 56 shown in FIG. 10 may be advanced out of a lumen in a catheter such as the colonoscope 14 through the side opening 36 of the overtube 12a to grasp the diverticulum 28 and invert it into the overtube as described previously to effect serosa-to-serosa closure, it being understood that as described above vacuum can be used to invert the diverticulum and that the overtube 12 with the open distal end can also be used. As also described above and as shown in FIG. 29, the diverticulum 28 can be inverted through a ligating loop 34 formed by control wires that draw the jaws over the tissue and that are slidably surrounded by a cinch tube 100, which causes the jaws to close as the control wires are drawn into the cinch tube.

In accordance with the embodiment shown in FIGS. 28-31, a tissue heating device 102 can be provided in the overtube 12a. FIGS. 28 and 29 show that the tissue heating device includes opposed arms 104, 106 that pivot, relative to each other at a pivot joint 108. The device 102 may be attached to a closed distal end of the overtube 12a by a return spring 110.

It may be appreciated in reference to FIGS. 28 and 29 that the inner surfaces 112 of the arms 104, 106 may be flat, and electrical leads can extend from the arms 104, 106 through the overtube 12a to a source of electricity that is external to the patient to heat the inner surfaces 112. The arms 104, 106 may be hollow so that they may be evacuated to further draw tissue into them. FIG. 30 shows that the cinch tube 100 can be advanced using, e.g., a push tube within the overtube 12a to ligate the diverticulum 28, and the heating device 102 likewise can be advanced to position the arms 104, 106 on opposite sides of the diverticulum 28. The heating device 102 may be advanced by any suitable means, e.g., using a push tube over an affixed wire or other means.

As the heating device 102 is advanced over the diverticulum 28, owing to the tensioning of the spring 110 the arms 104, 106 pivot toward each other, clamping the pursed colonic wall between them. The inner surfaces 112 are then heated to fuse clamped colonic wall portions together while cutting off the diverticulum 28. Instead of rigid arms 104, 106, a flexible conductive loop of, e.g., wire can be used and positioned around the stump, tightened, and energized to fuse clamped colonic wall portions together while cutting off the diverticulum 28. By “fuse” is meant tissue welding using principles of bipolar electrocautery, ultrasonic tissue welding, laser tissue welding, etc. in addition to heat fusion, in which case the arms 104, 106 are configured as electrocautery arms, ultrasonic transducers, laser emitters, etc.

FIGS. 32-36 show that one of the anchors 92 described above may be used in an alternate embodiment to resolve the diverticular area of the colonic wall after removal of the diverticulum 28 in which a non-pursed stump structure remains. More specifically, as figuratively shown in FIG. 32, the diverticulum 28 exists and as shown in FIG. 33 is inverted into the colonic lumen using the assembly 10 described above. As indicated by the arrow 114 in FIG. 34, the diverticulum 28 is then pulled forcefully using, e.g., the grasper 56 described previously to draw a first portion 116 of the colonic wall into an overlapping relationship with a second portion 118 of the colonic wall. It may be appreciated in reference to FIGS. 34 and 35 that the outer surface of the first portion 116 is flush against the inner surface of the second portion 118 in the overlapping relationship shown, i.e., that in this less preferred embodiment a serosa-to-mucosa mating is established. After the diverticulum 28 has been optionally removed as indicated by the arrow 120 in FIG. 36 using one of the above-described methods, one or more of the anchors 92 can be driven into the portions 116, 118 to hold them together as shown. The anchors 92 may be delivered using the structure described above in reference to FIGS. 22-27.

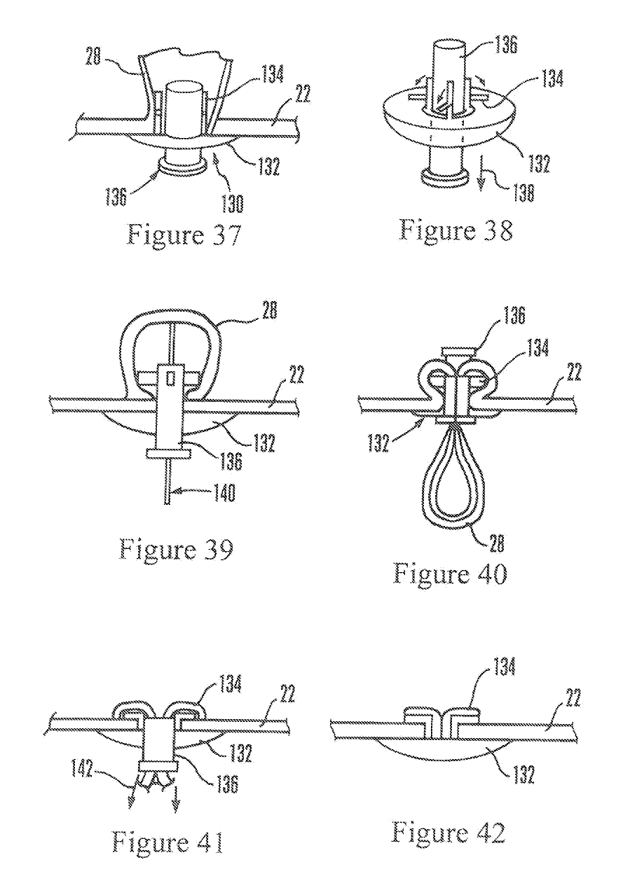

FIGS. 37-42 show that a plug 130 can be advanced through the colon wall 22 into the diverticulum 28 prior to inversion. In one non-limiting implementation the plug 130 can be positioned as shown in FIG. 37 by pushing it through the lumen of a catheter such as the overtube 12 or the catheter 14 or 16 (FIGS. 1-3) under, e.g., colonoscopic visualization.

In cross-reference to FIGS. 37 and 38, the plug 130, which is preferably made of biocompatible plastic, includes a half-dome-shaped stopper 132 that is radially larger than the remainder of the plug structure and also is radially larger than the opening of the diverticulum 28 as shown. Attached to the stopper 132 are plural elastic wings 134 that may be made of shape memory material, and passing centrally through the stopper 132 is a hollow cylindrical delivery tube 136. As shown in FIG. 37, the ends of the wings 134 opposite the stopper 132 are temporarily bonded using, e.g., a weak adhesive to the delivery tube 136 to hold the wings 134 in the straight configuration shown. When the delivery tube 136 is retracted into the overtube 12 as shown by the arrows 138 in FIG. 38, the bond breaks, freeing the wings 134 to assume their materially biased configuration in which they bend back parallel to or even against the outer surface of the colonic wall as shown in FIGS. 38 and 39. In this way, the colonic wall is sandwiched between the stopper 132 and wings 134.

Because the delivery tube 136 is hollow, irrigating fluid can be directed through the tube 136 to clean the diverticulum 28 prior to inversion. After cleaning, inversion may be effected by advancing a grasper 140 (FIG. 39) through the tube 136 to grasp the diverticulum 28 and retracting the grasper 140 to invert the diverticulum 28 back through the delivery tube into the colonic lumen as shown in FIG. 40 (and, thus, into the overtube 12 or 12a). The diverticulum 28 can then be cut off using any of the above-described methods and removed from the patient through the overtube.

Next, as shown by the arrows 142 in FIG. 41, the delivery tube 136 is retracted out of the stopper 132, releasing an inward mechanical force of the plug 130 on the inverted tissue, thereby effecting ligation. With the wings 134 preventing inward migration of the plug 130 into the colon, the plug 130 remains in place as shown in FIG. 42, ensuring that no leakage of air or fluids occurs from the colon to the peritoneal cavity and thus resolving the defect in the diverticular tissue. The wings 134 may be covered in an atraumatic material or configured as an atraumatic structure to avoid perforation and promote ingrowth. In addition to the hollow plug now being blocked by tissue, if desired glue or other substance may be infused into the hollow portion with the tissue to further block the plug.

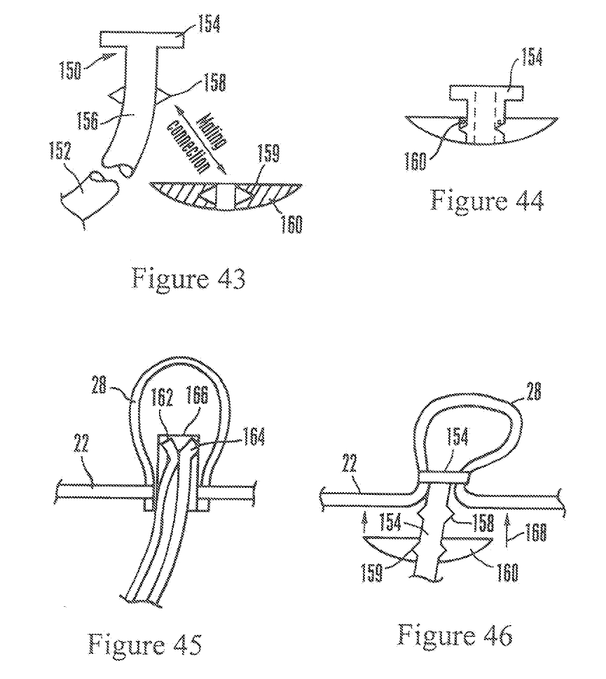

FIGS. 43-48 illustrate another plug embodiment, generally designated 150. The plug 150 is a silastic plug, i.e., a plug with rubber-like qualities of silicone which enable the parts to snap fit together.

As shown, the plug 150 includes an outer part 152 having a flange 154 that engages the outer colonic wall when deployed and a hollow shank 156 depending from the flange 154. The hollow shank 156 includes structure 158 such as a tapered ring that engages complementary structure 159 (such as a radial groove) on a hollow inner part 160 that is shaped substantially similarly to the stopper 132 discussed above and shown in FIGS. 37-42. The inner part 160 engages the inner surface of the colon.

As shown in FIG. 45, the flange 154 ultimately is established by a foldable flange 162, 164 that can be pulled into a delivery tube 166 to be nearly parallel with the hollow shank 156. The delivery tube is advanced into the diverticulum 28 as shown in FIG. 45 and then retracted, freeing the flange 164, 166 to assume the flange-like configuration shown in FIG. 46, thereby establishing the flange 154 on the outer surface of the colonic wall 22 as shown.

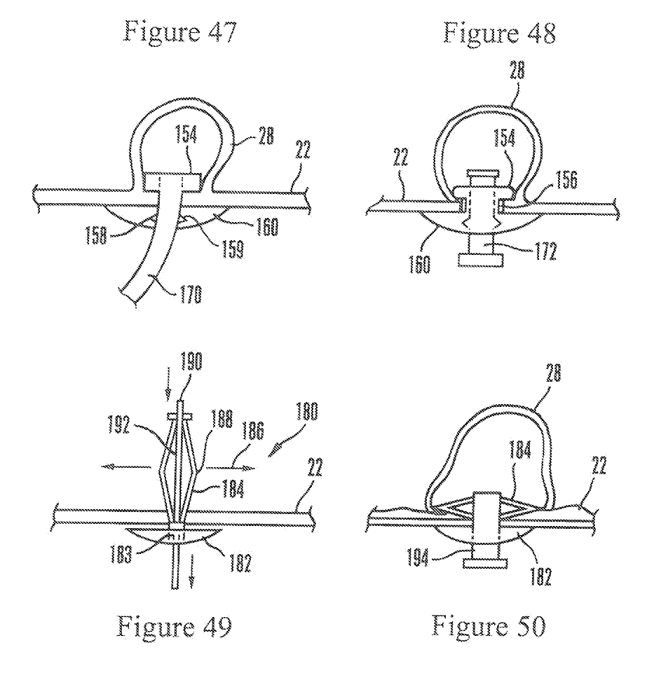

As also shown in FIG. 46 and as indicated by the arrows 168, the inner part 160 is advanced over the shank 154 until the interlocking structures 158, 159 of the outer part 150 and inner part 160, respectively, engage each other as shown in FIG. 47 to clamp the colonic wall between the flange 154 of the outer part 150 and the flat surface of the half dome-shaped inner part 160. The portion 170 of the hollow shank 156 that extends inwardly beyond the inner part 160 can be cut off, and a delivery tube 172 for flushing and inverting the diverticulum 28 can be advanced through the plug 150 as described above in relation to the plug 130 shown in FIGS. 37-42.

FIGS. 49 and 50 show an alternate plug 180 configured generally as a rivet. The plug 180 includes a stopper 182 with hollow central passageway 183, and the stopper 182 shown in FIGS. 49 and 50 is substantially identical in configuration and purpose to the stopper 132 shown in FIGS. 37-42.

Plural rivet legs 184 extend from the stopper 182 and as indicated by the arrows 186 in FIG. 49, are biased to bend radially outward at respective, bend joints 188 to assume the flattened, radially enlarged configuration of FIG. 50. To facilitate advancing the legs 184 through the diverticular defect into the diverticulum (not shown in FIG. 49), the ends of the legs 184 opposite the stopper 182 are connected to a rivet cap 190, and a push rod 192 is advanced into the rivet cap 190 and is pushed into the diverticulum to move the legs 184 to the relatively straight configuration shown in FIG. 49. In this configuration the legs 182 are moved through the colonic wall into the diverticulum, at which point the push rod 192 is retracted, allowing the legs 184 to assume the configuration shown in FIG. 50, clamping the colonic wall 22 between the stopper 182 and the legs 184 as shown. A delivery tube 194 is then advanced through the passageway 183 of the stopper 182 for flushing and inverting the diverticulum 28 through the plug 180 as described above in relation to the plug 130 shown in FIGS. 37-42.

With the above disclosure in mind, it may now be appreciated that the present invention affords the following advantages. A diagnostic procedure (imaging) and a therapeutic procedure (inversion and removal) are combined in one procedure. No incisions of the skin and muscular layers need be made, since the diverticulum is reached through a natural orifice. Likewise, general anesthesia is not required because the procedure can be done similar to polypectomy. Moreover, the patient suffers comparatively less pain after the procedure. Additionally, the risk of peri-interventional infection is reduced because the lumen of the bowel is not opened. Also, costs are reduced because a sterile operating room and hospitalization are not required to effect the procedure. Repair of the intestinal wall defect is done to prevent reoccurrence of the diverticulum.

While the particular SYSTEMS AND METHODS FOR ENDOSCOPIC TREATMENT OF DIVERTICULA are herein shown and described in detail, it is to be understood that the subject matter which is encompassed by the present invention is limited only by the claims. For example, the diverticulum 28 may be ablated through the overtube 12, and in lieu of being removed may be glued or bricked off. Or, the diverticulum may be covered and/or excised without inverting. The diverticulum may be desiccated by cautery, laser, chemical, cryogenics to make it slough off harmlessly. The defect may be plugged with a mesh or plug as discussed above. The process can involve anastomosing the defect closed, as well as the use of an implant overlay of the defect similar to a hernia treatment. The bowel layers may be folded over the defect to make multiple layer closure. Tissue growth factors can be infused through the overtube 12 to better heal the site, as can be antibiotics prior to removal. Moreover, radiopaque clips can be implanted through the overtube 12 and left in pace to facilitate monitoring the site for future recurrences of diverticular disease.

1. A method for treating a diverticulum formed in an intestinal wall of a patient, comprising:
advancing a tubular assembly into the intestine through a natural orifice to the site of the diverticulum;
inverting the diverticulum into the assembly; and
resolving a defect area associated with the diverticulum at least in part using the assembly by attaching one portion of the muscular wall of the intestine to another portion of the muscular wall.

2. The method of claim 1, wherein the defect area is resolved by drawing the diverticulum into the assembly, removing the diverticulum, and holding together intestinal wall tissue serosa to serosa.

3. The method of claim 1, wherein the intestinal wall tissue is sutured to hold it together serosa to serosa.

4. The method of claim 1, wherein the intestinal wall tissue is stapled to hold it together serosa to serosa.

5. The method of claim 1, wherein the intestinal wall tissue is held together using a barbed anchoring device.

6. The method of claim 1, wherein the intestinal wall tissue is held together serosa to serosa at least in part by clamping the wall between opposed elements of a tissue heating device and heating the elements to fuse clamped wall portions together.

7. The method of claim 1, wherein the defect area is resolved by disposing a cover over the defect area and adhering the cover to the intestinal wall.

8. The method of claim 1, wherein the defect area is resolved by plugging the defect area.

9-24. (canceled)

25. The method of claim 1, wherein the tubular assembly includes an overtube the inner surface of which is coated with a lubricious material to ensure smooth advancement of the overtube over a colonoscope.

26. The method of claim 1, wherein the tubular assembly includes an overtube containing a colonoscope, and the overtube and colonoscope are advanced together toward the diverticulum.

27. The method of claim 1, wherein the diverticulum is on the small intestine and the natural orifice is the mouth.

28. The method of claim 1, wherein the diverticulum is on the large intestine and the natural orifice is the anus.

29. A method for treating a diverticulum formed in an intestinal wall of a patient, comprising:
advancing a tubular assembly into the intestine through the mouth of the patient to the site of the diverticulum in the small intestine;
inverting the diverticulum into the assembly; and
resolving a defect area associated with the diverticulum at least in part using the assembly by attaching one portion of the muscular wall of the intestine to another portion of the muscular wall.

30. The method of claim 29, wherein the defect area is resolved by drawing the diverticulum into the assembly, removing the diverticulum, and holding together intestinal wall tissue serosa to serosa.

31. The method of claim 29, wherein the intestinal wall tissue is sutured and/or stapled to hold it together serosa to serosa and/or is held together using a barbed anchoring device and/or by clamping the wall between opposed elements of a tissue heating device and heating the elements to fuse clamped wall portions together.

32. The method of claim 29, wherein the defect area is resolved by disposing a cover over the defect area and adhering the cover to the intestinal wall.

33. The method of claim 29, wherein the defect area is resolved by plugging the defect area.

34. The method of claim 29, wherein the tubular assembly includes an overtube the inner surface of which is coated with a lubricious material to ensure smooth advancement of the overtube over a colonoscope.

35. A method for treating a diverticulum formed in an intestinal wall of a patient, comprising:
advancing a tubular assembly into the intestine through the anus of the patient to the site of the diverticulum in the large intestine;
inverting the diverticulum into the assembly; and
resolving a defect area associated with the diverticulum at least in part using the assembly by attaching one portion of the muscular wall of the intestine to another portion of the muscular wall.

36. The method of claim 35, wherein the defect area is resolved by disposing a cover over the defect area and adhering the cover to the intestinal wall or by plugging the defect area.
